(19) United States
(12) Patent Application Publication
(10) Pub. No.: US 2026/0137378 A1
(43) Pub. Date: May 21, 2026

(54) METHODS AND DEVICES FOR TRANSXIPHOID ACCESS TO THE MAMMARY ARTERIES

(71) Applicant: Andy C. Kiser, Greenville, NC (US)

(72) Inventor: Andy C. Kiser, Greenville, NC (US)

(21) Appl. No.: 19/443,549

(22) Filed: Jan. 8, 2026

Related U.S. Application Data
(62) Division of application No. 17/870,956, filed on Jul. 22, 2022.
Application No. 17/870,956 is a division of application No. 16/438,919, filed on Jun. 12, 2019, now abandoned.
(60) Provisional application No. 62/683,856, filed on Jun. 12, 2018.

(51) Int. Cl. A61B 17/02; A61B 1/00; A61B 1/04; A61B 1/05; A61B 1/06; A61B 17/00; A61B 17/04; A61B 17/32; A61B 17/34

(52) U.S. Cl. CPC A61B 17/02; A61B 1/00094; A61B 1/04; A61B 1/05; A61B 17/0206; A61B 17/32; A61B 1/0661; A61B 2017/00243; A61B 2017/0237; A61B 2017/0287; A61B 17/0469; A61B 17/3423

(57) ABSTRACT

A trans-xiphoid procedure for gaining entry to the chest cavity of a patient, sometimes also referred to herein as a “TRAX” procedure. The procedure may be used for, among other things, mobilization of the mammary arteries and performing coronary artery bypass surgery in which the mammary artery of the patient or other conduit is joined to a coronary artery of the patient, such as the left anterior descending (LAD) coronary artery.

### RELATED APPLICATIONS

The present application is a divisional of U.S. patent application Ser. No. 17/870,956, filed Jul. 22, 2022, which is a divisional of U.S. patent application Ser. No. 16/438,919, filed Jun. 12, 2019, which claims priority to U.S. Provisional Application No. 62/683,856, filed Jun. 12, 2018, the disclosures of which are incorporated herein by reference in their entirety.

### FIELD OF THE INVENTION

The present invention relates to methods and devices for trans-xiphoid access to the thorax, including direct access to the coronary arteries and to the left and right mammary arteries for mobilization and harvesting for coronary bypass or other revascularization procedures.

### BACKGROUND

Traditionally, access to the mammary arteries has been limited to direct surgical access via thoracotomy or thoracoscopy, both full and partial. The revascularization of the coronary arteries with the mammary arteries is therapeutic for coronary artery disease, and mammary arteries may be harvested and used to revascularize other vessels as well.

A currently preferred type of thoracotomy for accessing the mammary arteries is the median sternotomy. The mammary arteries are located inside the chest cavity, alongside the borders of the sternum. During median sternotomy, the sternum is split in half and spread apart to provide ready access to the mammary arteries, for coronary bypass or other purposes. Once the procedure is complete, the sternum is re-joined with permanently indwelling wires (or other metal devices) to promote bone healing. Unfortunately, the mammary arteries are also important sources of blood supply to the sternum itself. When other sources of sternal blood supply are compromised (such as in patients with diabetes, obesity, obstructive lung disease, radiation exposure, etc.) and the mammary artery is used for coronary revascularization, the re-joined sternum may not heal properly, resulting in sternal non-union or, in severe situations, infection leading to removal of the sternum. Surgical alternatives which avoid such damage to the sternum would be advantageous.

Others techniques for accessing the coronary arteries by cutting between the ribs still involve a painful recovery for the patient (see, e.g., U.S. Pat. No. 7,909,846 to Taylor et al.). Indeed, pain after thoracotomy is considered among the most severe experienced by patients after surgery. See, e.g., P. Gerner, Post-thoracotomy Pain Management Problems, Anesthesiol. Clin. 26(2):355-vii (2008). And, since chest pain of this type can discourage regular breathing and coughing, the problem of lung complications such as pneumonia can be serious. Hence, there is a need for devices and methods to access to the mammary arteries without cutting through the chest wall so that risk to the patient and trauma resulting from the surgery can be minimized.

### SUMMARY

A first aspect of the present invention is a trans-xiphoid procedure for gaining entry to the chest cavity of a patient, sometimes also referred to herein as a “TRAX” procedure. The procedure may be used for, among other things, mobilization of the mammary arteries and performing coronary artery bypass surgery in which the mammary artery of the patient or other conduit is joined to a coronary artery of the patient, such as the left anterior descending (LAD) coronary artery.

The progressive dissection of the mammary artery of the patient is performed by liberating the distal end of one or both internal mammary arteries at the inferior costal margin of the patient. Upon elevation of the patient's sternum after (optional) xiphoid removal, the distal end of the mammary artery is identified and divided after systemic anticoagulation. Gentle traction to the distal end of the mammary artery is provided by securing the end of the mammary artery pedicle with a large elastic band loop which is then secured to a band hook on the exposure device at a series of graded retraction positions on the elastic band loop. With the use of the suction device, the mammary dissecting device, cautery, high frequency coagulation (described by U.S. Pat. No. 5,846,236 to Lindenmeier and by US Publication No. 2008103495 to Mihori) and hemostatic clips the mammary artery is dissected free from the chest wall from distal to proximal to the level of the subclavian vein using general cardiothoracic surgical and endoscopic vessel harvesting techniques. Upon total liberation of one or both mammary arteries, the mammary artery exposure and dissection devices are removed and the pericardium is opened and retracted using the button described herein. The anastomosis to the coronary artery is performed with this pericardial retraction and with the assistance of the elevator described herein using generally accepted cardiothoracic surgical techniques, with or without cardiopulmonary support.

Currently, coronary bypass is a difficult procedure to perform without a sternotomy. Less invasive coronary bypass requires extensive training, may require expensive robotics, and is not without significant risk to the patient (Ikeda C, et al., Harvesting bilateral internal thoracic arteries using a novel subxiphoid approach versus the conventional lateral thoracic approach-results of an experimental study. J. Thorac. Cardiovasc. Surg. 148:461-7 (2014)). Access to the left anterior descending artery during percutaneous stent placement is feasible, but long-term results are better when the left internal mammary artery is used to bypass the left anterior descending artery. Performing this surgical bypass procedure via the trans-xiphoid approach without sternotomy should decrease morbidity and decrease hospital admission.

US Patent Application Publication No. US 2014/0358219 to Nambiar describes a method for thoracic artery harvesting in which the sternum is elevated, but access to the chest cavity is still by an incision through the chest wall (a “mini-thoracotomy”)

U.S. Pat. No. 7,219,671 to Benetti describes methods for coronary artery bypass, that are the to avoid opening the chest wall, but these still require piercing the chest wall with a trocar (see FIG. 11). Similarly, U.S. Pat. Nos. 6,613,069 and 6,482,151, both assigned to Heartport, show piercing of the chest wall with access ports.

The present invention is explained in greater detail in the drawings herein and the specification set forth below.

### BRIEF DESCRIPTION OF THE DRAWINGS

FIG. 1 schematically illustrates the steps of a surgical procedure carried out with the methods and instruments described herein.

FIG. 2 illustrates the chest and abdomen of a subject and the location of a trans-abdominal incision made to carry out the TRAX procedure.

FIG. 3 illustrates a bed-mounted retractor and access system, or “exposure device,” to lift the sternum and chest wall and enhance exposure within the trans-abdominal incision when carrying out the TRAX procedure.

FIG. 4 illustrates the device of FIG. 3, further including a mounting element connected through an adjustable frame to a surgical table, and positioned over a patient.

FIG. 5 is top perspective view of the device of FIG. 3.

FIG. 6 is a back view of a device of FIG. 3.

FIG. 7 is a schematic illustration of an elastic mammary artery retractor band.

FIG. 8 is a schematic illustration of an elastic mammary artery retractor band of FIGS. 7, secured to the distal end of a mammary artery.

FIG. 9 is a perspective view of a suction device useful for separating a mammary artery from the interior chest wall during a TRAX procedure.

FIG. 10 is a back perspective view of the distal end of a suction device of FIG. 9, with the resilient cushioning outer shell shown transparent.

FIG. 11 is a front perspective view of the distal end of a suction device of FIG. 9, with the resilient cushioning outer shell again shown transparent.

FIG. 12 is a front view of the suction device of FIG. 11.

FIG. 13 is a top, cut-away, view of a first embodiment of a cushion elevator useful for positioning the heart for connection of a coronary artery to a mammary artery during a TRAX procedure.

FIG. 14 is a top, cut-away, view of a second embodiment of a cushion elevator useful for positioning the heart for connection of a coronary artery to a mammary artery during a TRAX procedure.

FIG. 15 is a top, cut-away, view of a third embodiment of a cushion elevator useful for positioning the heart for connection of a coronary artery to a mammary artery during a TRAX procedure.

FIG. 16 is a top, cut-away, view of a fourth embodiment of a cushion elevator useful for positioning the heart for connection of a coronary artery to a mammary artery during a TRAX procedure.

FIG. 17 is a side schematic view of a cushion elevator, as positioned beneath the heart and back chest wall to aid in positioning the heart for connection of a coronary artery to a mammary artery during a TRAX procedure.

FIG. 18 is a first perspective view of a suture retainer device useful for, among other things, temporarily securing a suture while connecting a coronary artery to a mammary artery during a TRAX procedure.

FIG. 19 is a second perspective view of the suture retainer device of FIG. 18.

FIG. 20 is a top view of the suture retainer device of FIG. 18.

FIG. 21 is a bottom view of the suture retainer device of FIG. 18.

FIG. 22 is a side view of the mammary dissecting device with the dissecting knife retracted, with the dissecting knife extended and with a cautery-type device extended.

FIG. 23 is a view of the mammary artery and veins through the clear solid nose cone via an endoscope within the mammary dissecting device.

### DETAILED DESCRIPTION

In this specification and the appended claims, the singular forms “a,” “an” and “the” include plural referents unless the context clearly dictates otherwise. Thus, for example, the term “a lumen” is intended to mean a single lumen or a combination of lumens, “a fluid” is intended to mean one or more fluids, or a mixture thereof.

### 1. Definitions

Subjects or patients on whom the invention may be carried out are, in general, mammalian subjects, particularly human subjects, but also including animal subjects (dogs, cats, horses, cattle, sheep, goats) for veterinary purposes. Subjects may be male or female and may be of any age, including neonate, infant, juvenile, adolescent, adult, and geriatric subjects.

“Proximal” and “distal” as used herein refer to direction closer to and away from, respectively, an operator (e.g., surgeon, physician, nurse, technician, etc.) who would insert a device (e.g., an instrument of the present invention) into the patient, such that the distal end of the device is the end that is inserted inside a patient's body, while the end outside the patient's body would be the proximal end.

“Endoscope” as used herein is a small rigid or flexible tube with a light and lens that may be used to view an organ or body part via a cannula or a portal or a trocar.

“Epicardial surface” as used herein is the outer surface of the heart. The term epicardial surface is also used to refer to the innermost of the two layers of pericardium.

“Endocardium” as used herein is the membrane that lines the cavities of the heart and forms part of the heart valves—i.e., the innermost layer of the heart.

“Pericardial surface” as used herein is the outer surface of the pericardial sac. The pericardium is a double-walled sac that contains the heart and the roots of the great vessels.

“Pleural surface” as used herein is the outer surface of the lung (visceral pleura) and the inside of the chest wall (parietal pleura). The pleura is a thin membrane sac that covers the structures in the left and right thoracic cavities respectively.

### 1. Overview of TRAX Procedure and TRAX Mammary Harvest

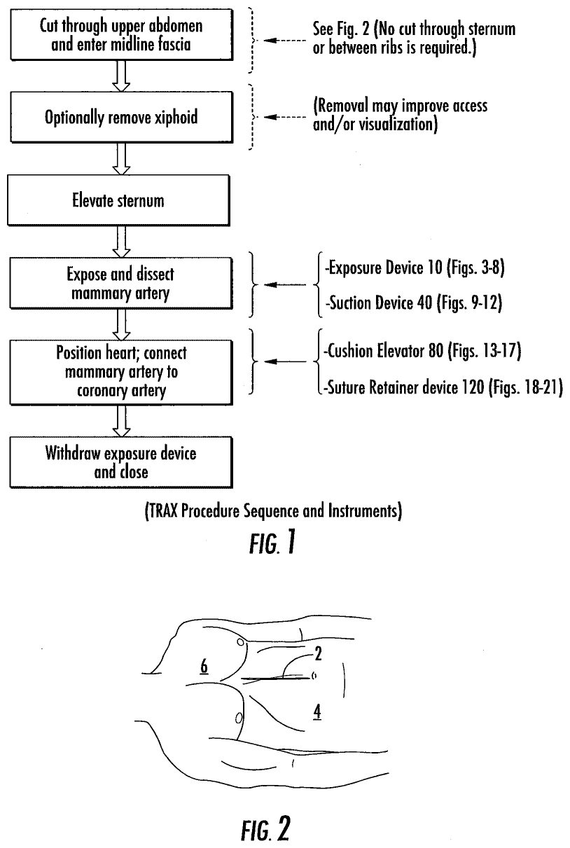

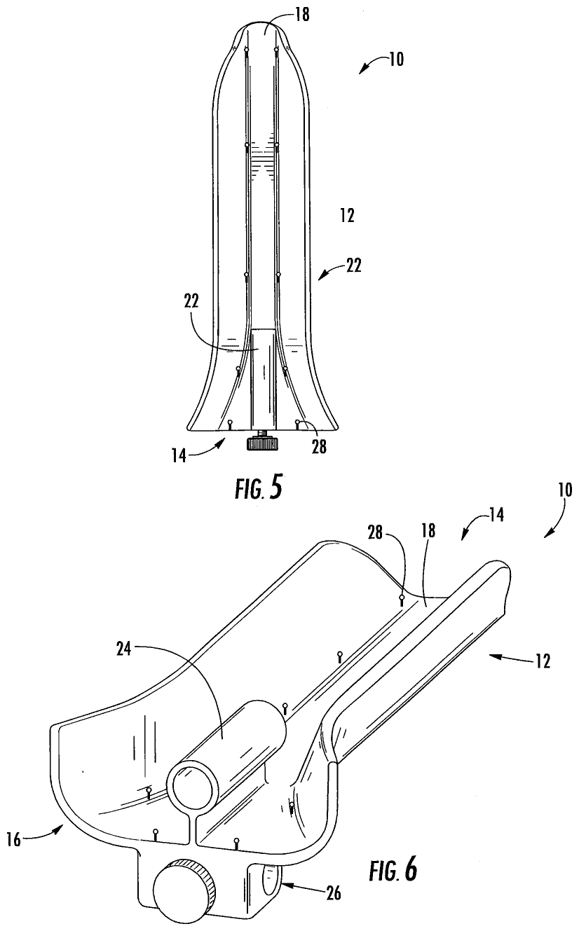

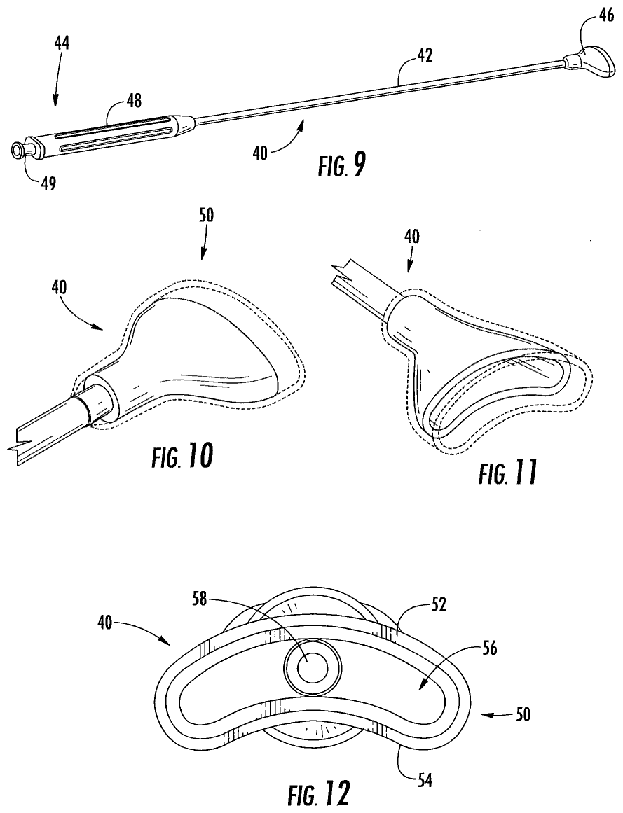

As noted above and schematically illustrated in FIGS. 1-2. In the TRAX procedure, an incision 2 is made through the upper portion of the patient's abdomen 4, and a further incision is made through the midline fascia but not necessarily into the peritoneum. No cut through the sternum, or cut between the ribs, is required. Next, the xiphoid process is optionally removed to improve visualization of the interior chest cavity, and the sternum 6 of the patient is preferably elevated, preferably with an exposure device such as described below and in FIGS. 3-8, to provide access to one or both mammary arteries on the interior portion of the sternum, and to the heart itself. The mammary artery is progressively dissected or separated from the interior chest wall or sternum, preferably aided with the suction device as described below and in FIGS. 9-12.

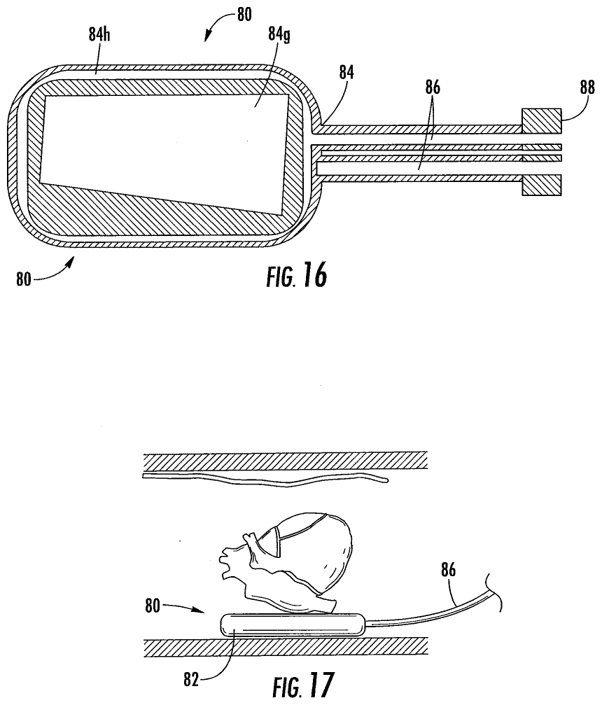

Once a sufficient segment of mammary artery is dissected free, that mammary artery is then joined to the coronary artery in need of bypass (such as the left anterior descending coronary artery of the subject) by conventional anastomosis techniques. To aid in positioning the heart for the anastomosis, a cushion elevator such as described below, and in FIGS. 13-17, may be placed beneath the heart to lift it upwards towards the incision.

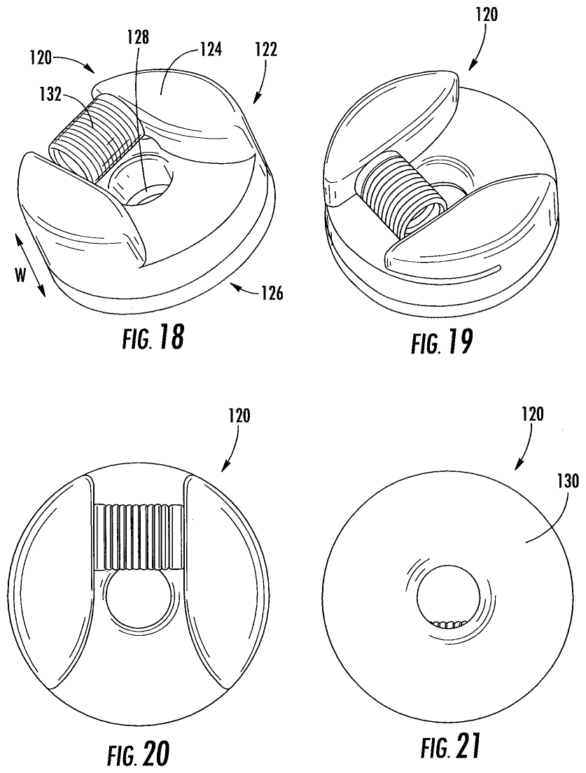

Exposure may be additionally aided by fastening a retention suture attached to the pericardium at various locations to a suture retainer device as described below, and in FIGS. 18-21 herein. The suture retainer device may be secured to a surgical drape, a surgical frame or the like close to the entry incision, which suture retainer provides a convenient tool for temporarily securing a free end of a suture as portions of the procedure are carried out.

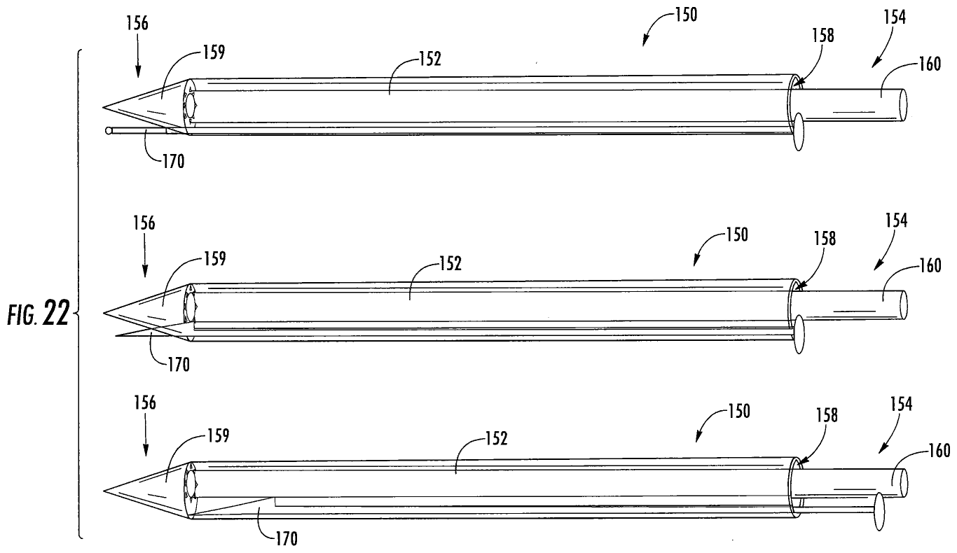

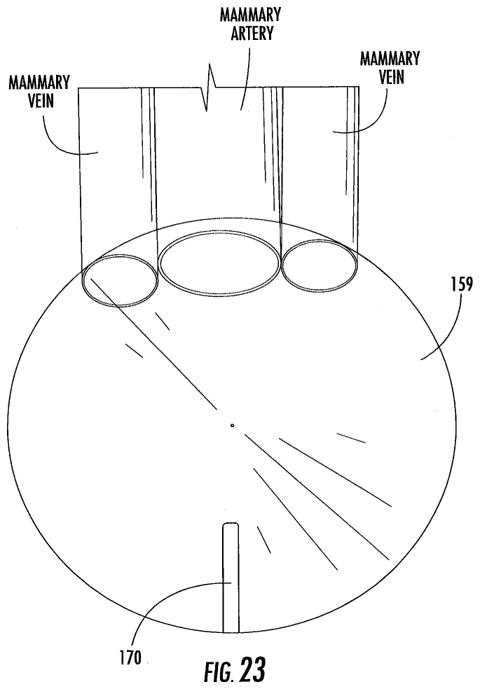

Dissection of the mammary arteries may be additionally aided by using the mammary artery dissecting device described below, and in FIGS. 22-23. The fascia overlying the mammary arteries and the branches of the mammary artery may be divided using the mammary dissecting device to aid in liberation of the mammary from the internal chest wall and divide/control branches of the mammary artery going to the chest wall.

During the procedure, the patient's lungs stay inflated when the pericardium or the pleural spaces are entered because the patient is under general endotracheal anesthesia with positive pressure lung ventilation. The pressure of the ventilator keeps the lungs inflated and, as long as there is no defect made in the surface of the lung, the lungs will remain inflated once the operation is complete. Because, in the procedures described herein, the procedures is performed over the diaphragm, not through the diaphragm, there is limited pulmonary compromise.

### 2. Mammary Exposure Device

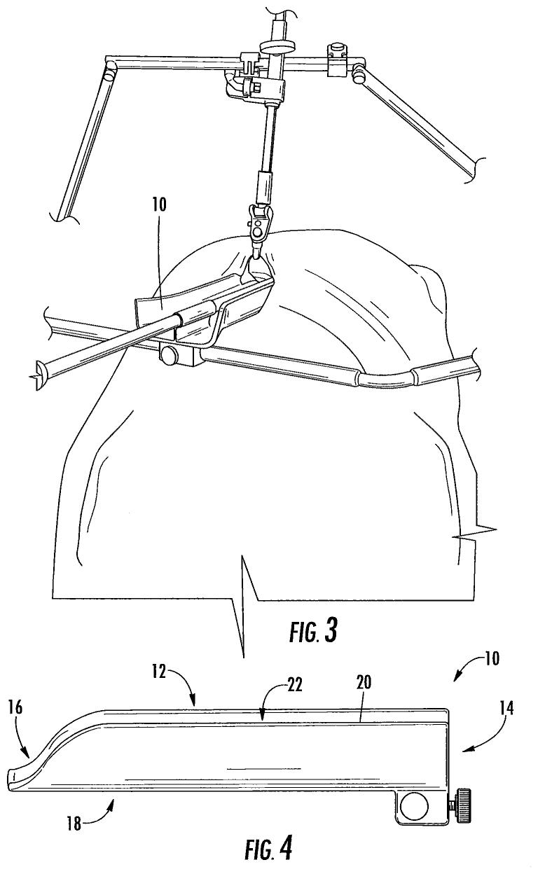

As shown in FIGS. 4-6, a mammary exposure and dissecting device 10 includes an elongate body member 12 having a distal (internal) end 14, a proximal (external) end 16, a bottom portion 18, and a pair of opposing side wall portions 20, the bottom and side wall portions 18, 20 together forming a top surface defining an elongate access channel 22 open through at least the proximal end.

The exposure device is securely attached to the patient's bed as shown in FIG. 3 and may have a configuration that includes an additional arm to generally lift the sternum or the ribs. Additional arms may be added or included to provide additional retraction or exposure.

The body member 12 is configured for insertion of the distal end 16 into the chest cavity of a subject through the subject's upper abdomen and through or above the diaphragm into a position overlying the subject's heart and underlying the subject's sternum, with both the right and left mammary arteries of the subject positioned above and accessible by a surgeon through the access channel 22.

In some embodiments an endoscope tunnel (or other mounting member or scope holder) 24 is positioned on the top surface, and (for example) configured to house either a 5 or 10 mm or other size endoscope which can be variably positioned along the length of the mammary exposure device (e.g., configured for extending an endoscope from proximal to distal through the access channel, and into the patient) so that the mammary arteries can be optimally illuminated and visualized. In certain embodiments the endoscope tunnel is elevated from the bottom member on a narrow platform or pedestal.

In some embodiments, a mounting element 26, such as a body with an aperture for mounting the device 10 onto a bar or mounting structure as shown in FIG. 3. It should be understood that any suitable mounting element may be used, such as a post or clamp.

In some embodiments there may be one or a plurality of anchor elements 28 on the top surface of the channel (e.g., hooks, open or closed loops (optionally with toggle elements), etc., preferably configured in a non-entrapping manner to avoid snagging or catching of fingers, instruments or the like), to attach elastic mammary artery retractor bands (discussed below), to the mammary exposure device so that the mammary artery may be secured to the device by the stretchable bands with stretching resistance or retracting tension. In certain instances, stretchable or elastic bands may be attached to the sides of the device, or at anchor elements arranged in spaced apart variable locations on the top surface from proximal to distal, and/or at variable locations at the proximal portion of the device, to provide a variable resistance platform to retract the mammary artery for optimal visualization.

The mammary exposure device 10 is mounted to the bed to secure position for stability. It may have an independent arm for connection to the bed that has an incorporated member to lift the sternum or may be associated with an existing commercially available device that mounts to the bed and lifts the sternum.

### 3. Elastic Mammary Artery Retractor Band

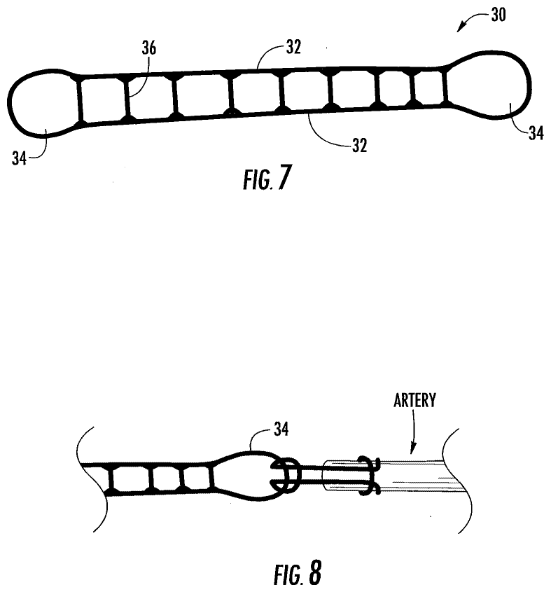

As noted above, the systems and apparatus described herein may also utilize an elastic mammary artery retractor band 30 as shown in FIGS. 7-8, which generally comprises: (a) a pair of elongate opposite elastic side members 32 having opposite end portions and an intermediate portions therebetween; (b) at least one elastic end loop member 34 connected to one of the opposite end portions and configured for securing to a mammary artery; (c) a plurality of elastic cross-members 36 interconnecting the elastic side members at spaced apart locations on the intermediate portions, each cross-member configured for securing to a stationary connecting member; and (d) optionally a second elastic end loop member connected to the other of the opposite end portions and configured for securing to a mammary artery.

As noted below, in some embodiments the end loop members are larger in size than the cross-members.

In some embodiments, the plurality of cross-members are uniformly sized and spaced from one another.

The retractor band may be integrally formed of an elastic polymer material (e.g., silicone, latex, etc.), and may be provided in sterile form sealed in a sterile package.

A non-limiting embodiment of such a retractor band is shown in FIGS. 7-8. In the example embodiment, the retractor band is configured as a larger loop segment, connected or formed with a series of smaller loop segments. In one iteration, the series of loops has large loop segments at both ends, with a series of small loop segments in between, all connected in series to one another with the larger loop at one end (or the larger loops at opposite ends), and configured so that they can be variably attached to the band hooks to provide graded resistance when connected to the mammary artery. The single loop may be the connection between the band containing the series of loops and the mammary artery. Additionally, the suction device can be utilized to assist the dissection of the mammary artery within the mammary exposure device.

### 4. Suction Device

Non-limiting examples of a suction device 40 useful for carrying out a TRAX procedure are given in FIGS. 9-12. In general, such a suction device 40 includes an elongate shaft 42 having a proximal (external) end 44 and a distal (internal) end 46, a handle 48 on the shaft proximal end 44, and an aspiration fitting 49 (e.g., a Luer fitting) on the shaft proximal end 44, and a suction fitting 50 on the shaft distal end. The device 40 may be of any suitable length, but typically will be no longer than 18 inches (50 cm) and no shorter than 6 inches (15 cm) for mammary harvest.

The suction fitting 50 may have an outwardly or convexly curved top surface portion 52, and/or an inwardly or concavely curved bottom surface portion 54. The suction fitting 50 includes an end terminal portion 56 with at least one distal suction opening 58 formed therein, with the suction opening in fluid communication with the aspiration fitting through the shaft 42.

In use, as a mammary artery is dissected from the sternum through the shaft the suction device may be inserted between the sternum and the freed mammary artery, and progressively advanced along the sternum. The convex top surface serves as a bumper against the sternum, and the concave bottom surface serves to guide and capture the freed mammary artery during dissection.

The shaft may be rigid, or may be flexible and resilient, depending upon the choice of the operator. As such, the shaft may be formed of any suitable material, including stainless steel and flexible polymers.

In the illustrated embodiment, as best shown in FIGS. 11-12, at least one suction opening is an elongate continuous suction opening (preferably arcuate in shape corresponding to the curvatures of the top and bottom surface portions), and is oriented substantially perpendicularly to the shaft. However, the at least one suction openings may comprise a series of openings, for example aligned along an arc, which arc is again oriented substantially perpendicularly to the shaft.

In some embodiments, the suction fitting includes: (i) a substantially rigid inner body portion (e.g., formed of a polymer material), and (ii) a resilient cushioning outer shell on the body portion. The outer shell may be formed of any suitable material, typically a cushioning polymer, and may be formed by any suitable technique, such as by overmolded on the (more rigid) inner body portion.

For the convenience of the operator, a suction control opening can be formed in either the handle or in the shaft adjacent the handle, with the suction control opening in fluid communication with both the fitting and the distal suction opening. By the operator alternately covering or uncovering the opening with a finger, the suction opening may be active of inactive (e.g., the vacuum bypassed through the control opening), so that the operator may modulate the vacuum force through the suction opening.

The suction device may be sterilized and sealed in a sterile package to provide the device in pre-packaged form ready for use.

### 5. Cushion Elevator

Non-limiting examples of a cushion elevator 80 useful for carrying out a TRAX procedure are given in FIGS. 13-17. In general, such a device 80 includes a flexible expandable cushion 82 configured for insertion inside the chest of a subject in a position underlying the heart, the cushion 82 having at least one expansion chamber 84a-84e formed therein. At least one flexible inflation line or inlet line 86 having a proximal (external) end 88 and distal (internal) end 89 is included, each inflation line connected to one of each of the at least one expansion chambers by the distal end and providing fluid communication therewith. Each inflation line is dimensioned to extend from the chest of a patient through the patient's diaphragm and out of an incision in the patient's abdomen when the cushion is positioned beneath the heart of the patient inside the patient's chest (as shown in FIG. 19), so that, by inflating the cushion by injecting fluid (e.g., sterile saline solution, sterile injectable oil such as vegetable oil, etc.) into an expansion chamber through the inflation line, the heart of the patient is lifted towards the sternum of the patient and joining of a mammary artery to a coronary artery in the patient is facilitated.

The device may include an external fluid supply (not shown) such as a syringe or bulb) operatively associated with the proximal end of each the inflation line. Where multiple chambers are included, each chamber may be provided with a dedicated fluid supply, or a single fluid supply with associated valve may be included.

The fluid supply or supplies may be removably connected to the inflation lines, or permanently affixed thereto. Where permanently affixed, the fluid supply (e.g., the syringes) may be pre-loaded with an amount of fluid matched to and appropriate for the expansion chamber with which they are associated. Also, when permanently affixed, the fluid supply may include a label, color code, or other identifier for the chamber with which they are associated (which identifier may alternatively be on the inflation line).

The cushion itself may be symmetric in shape, or may be asymmetric, with the asymmetry of the cushion matching the asymmetry of the dorsal surface of the typical patient's heart.

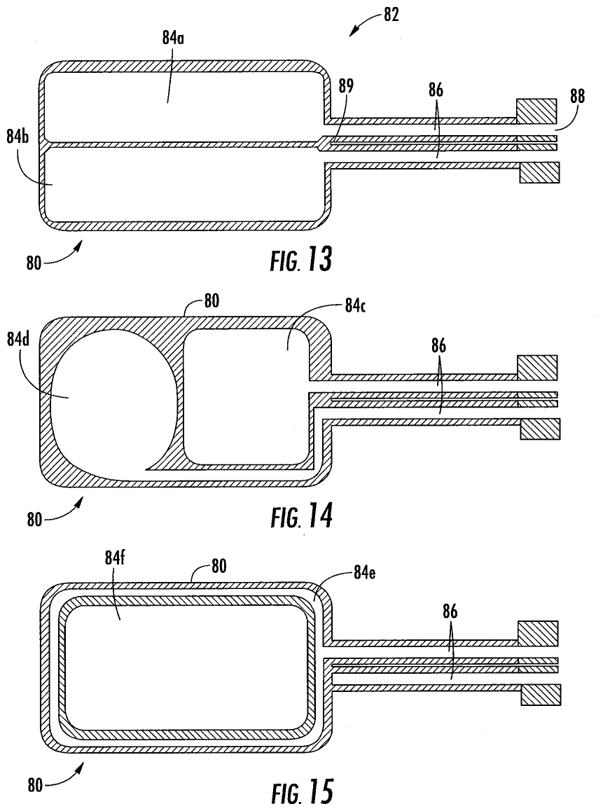

While, in the illustrated embodiments, two chambers are shown, it will be appreciated that additional chambers may be included. In addition, the chambers may be symmetric (as in FIG. 13) or asymmetric (as in FIG. 14), again with asymmetry matching the asymmetry of the dorsal surface of the typical patient's heart. The chambers may be arranged in a side-by-side manner as shown in FIGS. 13-14, or may be arranged in a concentric manner, as shown in FIGS. 15-16 (in some embodiments, with the outer chamber serving to create a capturing bumper for aligning the device with the patient's heart when inflated. Combinations thereof may also be employed (e.g., an outer “capturing” inflation chamber, as shown in FIGS. 15-16, surrounding two or more inner chambers, as shown in FIGS. 13-14).

In general, the cushion 82 is formed of at least one elastic polymer. The inflation lines are also formed of a flexible polymer, though in some embodiments a different polymer less susceptible to inflation. The cushion itself may be formed of multiple different polymers, as indicated in FIG. 16, to impart different elastic characteristics, and different inflation characteristics, to different regions of the elevator.

As with the suction device, the cushion elevator device may be sterilized and sealed in a sterile package to provide the device in pre-packaged form ready for use.

### 6. Suture Retainer Device

Non-limiting examples of a suture retainer device 120 useful for, among other things, carrying out a TRAX procedure are given in FIGS. 18-21. Such a device 120 generally includes a body 122 having a top portion 124, a bottom portion 126, a width dimension w, and a transverse channel 128 formed in the top portion 124, the transverse channel 128 and extending the entire width dimension of the body 122, an adhesive member 130 connected to the body bottom portion 126, and a suture retainer 132 connected to the body and positioned within the transverse channel.

In some embodiments, and as illustrated the transverse channel 128 includes an outwardly flared segment opposite the suture retainer (e.g., with the opening positioned therebetween) to aid in guiding suture into the transverse channel and the suture retainer.

Also, in some embodiments, and as illustrated, the body 122 has an opening formed therein, the opening extending from the top portion to the bottom portion, with the opening laterally aligned with and oriented substantially perpendicularly to the top transverse channel (e.g., with the opening substantially aligned with the center axis of the body). In some embodiments, the suture retainer is configured to partially overlaps or partially occludes the opening, so that, when suture is passed through the opening (as may be done in some but not all uses of the device), the suture may more easily engage the retainer.

In the illustrated embodiment, the suture retainer 132 is a coiled wire, but any suitable suture retainer may be used, including but not limited to combs, viscous gels, gums, etc. Also, while the illustrated embodiment is cylindrical or round in shape (when viewed from the top), it may be configured with any suitable cross-section, including oval and polygonal (e.g., square, hexagonal, octagonal, etc.). The suture retainers may optionally be provided in sets of multiple retainers, each with a different cross-section, to provide tactile identification associated with different sutures drawn therethrough.

A protective cover or sheet may be removably connected to the adhesive member, for removal just prior to use, to help preserve the adhesive properties of the adhesive member.

As with the suction device and the cushion elevator device, the suture retainer device may be sterilized and sealed in a sterile package to provide the device in pre-packaged form ready for use.

### 7. Mammary Dissecting Device

Non-limiting examples of a mammary dissecting device 150 useful for carrying out a TRAX procedure are given in FIGS. 22-23. In general, such a dissecting device includes an elongate shaft 152 having a proximal (external) end 154 and a distal (internal) end 156, a handle on the shaft proximal end, and a central opening 158 for the insertion of an endoscope 160 of appropriate size (1-10 mm). The endoscope 160 is inserted into the proximal (external) opening 158 and positioned in the distal (internal) end 156 where a dissecting tip 159 is present. The dissecting tip 159 can be uniformly or non-uniformly tapered, from proximal to distal. In this embodiment a single tapered tip is demonstrated. Pleural tapered tips may be included in some embodiments. The end of the tip, or tips, (which may be sharply pointed, bluntly pointed, blade shaped, etc.) may be aligned with, or offset from, the central axis of the elongate shaft 152. The dissecting tip 159 may be formed from a single material, such as a visually transparent rigid polymer, glass or sapphire, or formed from an assembly of materials (e.g., with a visually transparent material for a window through which the mammary artery may be visualized, which window is mounted in a frame formed of another material such stainless steel, which frame is configured to facilitate dissection of the mammary artery). The dissecting tip may be entirely solid, or may have a hollow region formed therein, although it is preferably shaped and connected to the shaft in a manner that prevents tissue, blood or other biological material from entering the central opening. In one non-limiting embodiment as discussed below, the dissecting tip is in the form of a cone.

In use, the dissecting tip is positioned between the mammary artery and the surrounding fascia and/or chest wall and, as the mammary dissecting device is advanced from proximal to distal (from external to internal) the graduated size of the tip expands the space between the mammary artery and the surrounding fascia and/or chest wall under endoscopic visualization.

The device (including shaft and tip) may be of any suitable length, but typically will be no longer than 18 inches (50 cm) and no shorter than 6 inches (15 cm) for mammary harvest.

The dissecting device may have a clamp, securement screw, sleeve, spring, or other attachment mechanism connected to the proximal (external) end of the shaft to secure the endoscope in place and limit motion of the endoscope during mammary dissection.

In use, as a mammary artery dissection device is freely rotated and advanced to separate the mammary artery from surrounding attachments. While dissecting the mammary artery from the sternum with the device, fascia surrounding the mammary artery may be encountered. These fascial attachments can be divided using the incorporated instrument, such as a knife or cautery device 170 (preferably contained in a second instrument lumen or elongate opening, which instrument lumen or opening is separate from the central opening that carries the endoscope) by advancing and retracting the knife or cautery device under endoscopic vision. The mammary artery is protected during fascia division by rotating the mammary dissecting device to position the mammary artery away from the knife or cautery device (for example, rotating 180° away). The dissecting device thus facilitates safe separation of the mammary artery from the knife and/or cautery. During mammary artery dissection using the mammary dissecting device, branches of the mammary artery may be encountered. These branches may be liberated for a safe distance from their mammary artery origin with the dissecting device to then be safely cauterized in a position away from (e.g. 180° away from) the mammary artery, under endoscopic visualization.

The shaft of the mammary dissecting device may be rigid, or may be flexible and resilient, depending upon the choice of the operator. As such, the shaft may be formed of any suitable material, including stainless steel and flexible polymers.

In the non-limiting illustrated embodiment, as shown in FIGS. 22 and 23, the mammary dissecting device is an elongate cylindrical structure with a continuous opening from the proximal end to the distal end which ends with a solid, clear dissecting tip. The solid clear tip enables separation of the mammary artery from surrounding associated structures under endoscopic vision through the clear cone. The knife and/or cautery device can be advanced and retracted at the distal (internal) end by action at the proximal (external) end and can be energized by an on/off switch external to the patient. This energy may be mono-polar or bi-polar radiofrequency or high frequency sonic cautery, or any other suitable cauterizing energy source.

The foregoing is illustrative of the present invention, and is not to be construed as limiting thereof. The invention is defined by the following claims, with equivalents of the claims to be included therein.

What is claimed is:

1. A suction device useful for separating the mammary arteries of a subject from the internal chest wall of the subject, comprising:
an elongate shaft having a proximal end and a distal end configured for placement internally in a subject during use;
a handle on the shaft proximal end;
an aspiration fitting on the shaft proximal end; and
a suction fitting on the shaft distal end, the suction fitting having a outwardly or convexly curved top surface portion and an inwardly or concavely curved bottom surface portion, an end terminal portion, and at least one distal suction opening in the end terminal portion, the suction opening in fluid communication with the aspiration fitting through the shaft.

2. The suction device of claim 1, wherein the elongate shaft is rigid or flexible.

3. The suction device of claim 2, wherein the at least one suction opening is an elongate continuous suction opening oriented substantially perpendicularly to the shaft.

4. The suction device of claim 1, wherein the at least one suction opening comprises a plurality of separate suction openings aligned along an arc, which arc is oriented substantially perpendicularly to the shaft.

5. The suction device of claim 1, wherein the suction fitting comprises: (i) a rigid inner body portion; and (ii) a resilient cushioning outer shell on the body portion.

6. The suction device of claim 1, further comprising a suction control opening formed in either the handle or in the shaft adjacent the handle, the suction control opening in fluid communication with both the aspiration fitting and the distal suction opening.

7. The suction device of claim 1, in sterile form sealed in a sterile package.

8. A suture retention device, comprising:
a body having a top portion, a bottom portion, a width dimension, and a transverse channel formed in the top portion, the transverse channel extending the entire width dimension of the body;
an adhesive member connected to the body bottom portion, with the channel extending therethrough;
a suture retainer connected to the body and positioned within the transverse channel.

9. The retention device of claim 8, wherein the suture retainer comprises a coil, a comb, a viscous gel, or a gum.

10. The suture retention device of claim 8, the body having an opening formed therein, the opening extending from the top portion to the bottom portion, with the opening laterally aligned with and oriented substantially perpendicularly to the top channel with the opening substantially aligned with the center axis of the body.

11. The suture retention device of claim 8, further comprising a protective cover removably connected to the adhesive member.

12. The suture retention device of claim 10, wherein the transverse channel includes an outwardly flared segment opposite the suture retainer with the opening positioned therebetween to aid in guiding suture into the transverse channel and the suture retainer.

13. The suture retention device of claim 8, wherein the body is cylindrical or polygonal.

14. The suture retention device of claim 8, in sterile form sealed in a sterile package.

15. A mammary artery dissecting device, comprising:
(a) an elongate shaft having a proximal end, a distal end, and a central opening formed therein, sand central opening configured to removably receive an endoscope for visualizing a mammary artery during dissection thereof;
(b) a dissection tip connected to the distal end, the dissection tip including a transparent portion configured to form a visualization window therein through which a mammary artery may be visualized with an endoscope in the central opening;
(c) at least one elongate cutting instrument and/or elongate cauterizing instrument axially aligned with and slideably connected to the elongate shaft, each instrument configured for slideably advancing the instrument out of the proximal end to facilitate cutting and/or cauterizing of tissues or vessels during dissection of a mammary artery, and then slideably retracting the instrument back into the proximal end when cauterizing or cutting is completed.

16. The dissecting device of claim 15, wherein the shaft is configured for rotation during dissection of a mammary artery to thereby position a cutting and/or cauterizing instrument away from the mammary artery during use.

17. The dissecting device of claim 15, wherein the dissecting tip is conical.

18. The dissecting device of claim 15, wherein the dissecting tip is formed from a single, visually transparent, material.
